(19) United States
(12) Patent Application Publication
(10) Pub. No.: US 2010/0292631 A1
(43) Pub. Date: Nov. 18, 2010

(54) Control flow device

(75) Inventor: Hugo Ross Holden, Queensland (AU)

Correspondence Address: JONES DAY, 222 EAST 41ST ST, NEW YORK, NY, 10017, US

(73) Assignee: Opto Global Holdings Pty Ltd., Thebarton, Adelaide, South Australia (AU)

(21) Appl. No.: 12/308,406

(22) PCT Filed: Jun. 18, 2007
(86) PCT No.: PCT/AU2007/000845

§ 371 Date: Jul. 30, 2010

(30) Foreign Application Priority Data
Jun. 16, 2006 (AU) 2006903273

(51) Int. Cl. A61F 9/007; A61M 1/00

(52) U.S. Cl. 604/22; 604/31

(57) ABSTRACT

The present invention relates to a flow control device for phacoemulsification procedures. The flow control device is valve which limits the vacuum surge that can occur when an occlusion in the phacoemulsification aspiration line is dislodged. The flow control device comprises; a body; a chamber formed therein; at least one inlet in communication with the chamber; and at least one outlet in communication with the chamber; wherein the chamber has at least a first portion and at least a second portion that are substantially divided by a member where the member has at least one restricted flow passage, and wherein the member is adapted to adjust a flow rate through the body by adjusting a flow resistance through the body responsive to the flow rate through the restricted flow passage within the device.

### INCORPORATION BY REFERENCE

The present application is related to and claims priority from Australia provisional application No. 2006903273, filed on 16 Jun. 2006 This provisional application is herein incorporated by reference in its entirety

### FIELD OF THE INVENTION

This invention relates to devices, methods, and systems that maintain acceptable flow rates and acceptable pressures in ophthalmic procedures such as Phaco emulsification. This invention also relates to controlling transient flow disturbances and transient pressures in such devices, method and systems. The invention also relates to a valve for controlling the rate of flow of a fluid in a tube, especially an aspiration tube used for aspiration of tissues and fluids in an ophthalmic procedure such as phaco emulsification.

### BACKGROUND OF THE INVENTION

The fluid delivery and control systems for state of the art phaco-emulsification cataract surgery have been compromised from the outset of these inventions by problems. These problems have resulted in unstable pressures in the eye's anterior chamber, and therefore unstable anterior chamber geometry at times during cataract surgery. The problems adversely affect the operating environment within the eye. This instability may result in the collapse of the eye's anterior chamber, and this can result in damage to the eye's delicate tissues. Complications include lens capsule rupture, iris damage and corneal damage. Capsular rupture predisposes to other complications such as glaucoma, macula oedema and retinal detachment.

There are generally two major forms of instability.

First, the pressure in the anterior chamber may drop with steady flow due to fluid flow resistance in the irrigation pathway. Therefore if the flow rate is too high the anterior chamber can collapse. Typically a flow rate of 65 ml/min may collapse the anterior chamber with a standard set of disposables and a 70 cm irrigating bottle height. Phaco machines that use a Venturi principle for creating a vacuum are particularly problematic in this regard as the fluid flow rate cannot generally be well controlled with these machines because it depends primarily on the applied vacuum level which may be variable during the surgery.

Second, the pressure in the eye may drop transiently due to rapid fluid out-flow from the eye's anterior chamber into the probe needle and fluid aspiration pathway. This may occur because at times the probe needle may become occluded (by cataract debris) and the vacuum in the aspiration system rises to a high value.

Under these circumstances there is storage of energy in the compliant parts of the aspiration system (eg the aspiration tubing, pump tubing and vacuum sensor assembly) because they have had a significant vacuum on the interior of their structures. The exterior parts and walls of these compliant (elastic) structures are compressed by atmospheric pressure and energy is stored. When the occlusion breaks free at the needle, fluid is rapidly drawn into the probe needle and aspiration system, as the compliant structures expand back to their previously uncompressed geometry. The peak outflow of fluid can, for example, exceed 70 ml/min or can exceed 100 ml/min and collapse the anterior chamber. This is particularly observed with the use of peristaltic pump-based phaco machines. The phenomenon is known as a “post-occlusion surge”.

The problem of chamber collapse at high flow rates can be ameliorated by placing a fixed flow resistive device in the aspiration line to limit the flow rates to lower values. For Venturi-based and peristaltic pump based phaco machines, this requires the vacuum levels to be run at a higher value and prevents the flow rate from becoming excessive. However the disadvantage is that when the vacuum is at lower levels, as it is at times during the surgery, the flow rate is severely retarded. Fluid flow cools the ultrasound crystals and the needle they are connected to in the phaco probe, and therefore there is more needle heating and wound burn with lower flow rates. Also slow or low flow rates do hot encourage cataract debris to be aspirated and cleared from the eye's anterior chamber in a short period. The commercially available Cruise Control Device, which is basically a fixed flow resistor, is not the solution for those reasons.

There is a need to be able to control the flow rate of fluid in surgical procedures that involve aspiration of tissue and/or fluid, such as phaco emulsification.

### SUMMARY OF THE INVENTION

The present disclosure is directed to devices, methods, or systems that improve on the control of flow rate and pressure in ophthalmic procedures such as Phaco emulsification. Certain embodiments relate to devices, methods, and systems that maintain acceptable flow rates and acceptable pressures in ophthalmic procedures such as Phaco emulsification. Certain embodiments also relate to controlling transient flow disturbances and transient pressures in such devices, method and systems. Certain embodiments also relate to a valve for controlling the rate of flow of a fluid in a tube, especially an aspiration tube used for aspiration of tissues and fluids in an ophthalmic procedure such as phaco emulsification.

In certain embodiments there is provided a flow control device comprising: a body having; a chamber formed therein; at least one inlet in communication with the chamber; and at least one outlet in communication with the chamber; wherein the chamber has at least a first portion and at least a second portion that are substantially divided by a member where the member has at least one restricted flow passage, and wherein the member is adapted to adjust a flow rate through the body by adjusting a flow resistance through the body responsive to the flow rate through the restricted flow passage within the device.

In certain embodiments there is provided a flow control device comprising: a body having; a chamber formed therein; at least one inlet connected to the chamber; and

at least one outlet connected to the chamber; wherein the chamber has at least a first portion and a second portion that are divided by a member, and wherein the member is adapted to adjust a flow rate through the body by adjusting a flow resistance through the body responsive to the flow rate via an aperture within the device. The devices then behaves as a device where the overall flow resistance increases proportionally to the differential pressure between the inlet and the outlet of the device, so as to stabilize the flow rate in view of increasing pressure differentials between the inlet and outlet of the device.

In certain embodiments there is provided a flow control device comprising: a body having; a chamber formed therein; at least one inlet in communication with the chamber; and at least one outlet in communication with the chamber; wherein the chamber has at least a first portion and a least a second portion that are divided by a member where the member has at least one restricted flow passage, and wherein the member is adapted to adjust a flow rate through the body by being capable of alternating between a more flow resistance and a less flow resistance configuration in response to flow variations through the device.

In certain embodiments there is provided a flow control device comprising: a body having; a chamber formed therein; at least one inlet connected to the chamber; and at least one outlet connected to the chamber; wherein the chamber has at least a first portion and a second portion that are divided by a member, and wherein the member is adapted to adjust a flow rate through the body by being capable of alternating between a more flow resistance and a less flow resistance configuration in response to flow variations through the device.

In certain embodiments there is provided a flow control device comprising: a body having; a chamber formed therein; means for an inlet of fluid into the chamber; means for outlet out of fluid from the chamber; means for dividing the chamber where the chamber has at least a first portion and at least a second portion; means for restricting the flow of fluid between the at least a first portion and the at least a second portion; and means for adjusting a flow rate through the body responsive to a differential flow rate through the means for restricting the flow of fluid.

In certain embodiments there is provided a method of controlling flow rate through a device comprising the steps of: sensing a flow rate between an inlet and an outlet of a body; if the flow rate increases, adjusting a position of a member such that a flow resistance is increased; and if the flow rate decreases, adjusting the position of the member such that a flow resistance is decreased; whereby an increase in flow rate is countered by an increase in flow resistance and a corresponding mitigation of the increased flow rate, and a decrease in flow rate is countered by a decrease in flow resistance and a corresponding mitigation of the decreased flow rate.

In certain embodiments there is provided a system comprising: a surgical control console; an irrigation device; a surgical instrument for performing an surgical operation on an eye and connected to the irrigation device and the instrument is controlled by the console; an aspiration device connected to the surgical instrument for aspirating fluid and tissue from the eye to a collection receptacle associated with the aspiration device; and a flow control device connected between the aspiration device and the surgical instrument wherein the flow control device includes, a body having a chamber formed therein; an inlet connected to the chamber; and an outlet connected to the chamber; wherein the chamber is divided by a member into at least a first portion and a second portion; wherein the member is adapted to adjust a flow rate through the body by adjusting a flow resistance through the body responsive to a differential pressure between the inlet and the outlet or responsive to a flow rate passing via the device. In certain aspects the irrigation device, the surgical, aspiration device, and the flow control device of the system are inter connected with tubing to allow fluid to flow through the system as needed. In certain aspects the control flow device and tubing of the system are disposable. In certain aspects a set off directions are provided on how to use the control flow device with the rest of the disposable package.

In certain embodiments there is provided a flow control device comprising: a body having; a chamber formed therein; at least one inlet connected to the chamber; and at least one outlet connected to the chamber; wherein the chamber has at least a first portion and a second portion that are divided by a member, and wherein the member is adapted to adjust a flow rate through the body by being capable of alternating between a more flow resistance and a less flow resistance configuration in response to pressure variations applied to the device.

In certain embodiments there is provided a flow control device comprising: a body having; a chamber formed therein; at least one inlet in communication with the chamber; and at least one outlet in communication with the chamber; wherein the chamber has at least a first portion and a second portion that are substantially divided by a member, and wherein the member is adapted to adjust a flow rate through the body by being capable of alternating between a more flow resistance configuration and a less flow resistance configuration to control transient flow disturbances and maintain the flow within an acceptable flow rate range. In certain aspect, the member of these embodiments is adapted to have at least one aperture, oriface, or restrictive flow passage to adjust a flow rate through the body by adjusting a flow resistance through the body responsive. The devices then behaves as a device where the overall flow resistance increases proportionally to the differential pressure between the inlet and the outlet of the device, so as to stabilize the flow rate in view of increasing pressure differentials between the inlet and outlet of the device.

In other embodiments there is provided a flow control device comprising: a body having; a chamber formed therein; an inlet connected to a proximal end of the chamber and in communication with the chamber; and an outlet connected to a distal end of the chamber and in communication with the chamber; and means for adjusting a flow rate through the valve body responsive to a differential pressure between the inlet and the outlet.

In other embodiments there is provided a flow control device comprising: a body having; a chamber formed therein; an inlet connected to a proximal end of the chamber and in communication with the chamber; and an outlet connected to a distal end of the chamber and in communication with the chamber; and means for adjusting a flow rate through the valve body responsive to a differential flow rate between the inlet and the outlet.

In certain aspects the flow control device the member is subjected to a biasing force that causes the member to minimize the flow resistance until the flow rate exceeds a predetermined value. In some aspects this biasing force is a spring, plate or rod. In other aspects this the biasing force and the member are a diaphragm. In other aspects the flow control device member is a mechanically movable piston. In other aspects the device member is a solenoid operated movable piston.

In certain embodiments the flow control device has a differential pressure sensor disposed between the inlet and the outlet; and a controller coupled to the differential pressure sensor; wherein the member is a solenoid operated piston having a position controlled by the controller responsive to a sensed differential pressure. In other aspects the flow control device has a differential flow sensor disposed between the inlet and the outlet; and a controller coupled to the differential flow sensor; wherein the member is a solenoid operated piston having a position controlled by the controller responsive to a sensed differential flow.

In other aspects the flow control device outlet has at least one variable resistance orifice and at least one flow bypass orifice in communication with the outlet. In other aspects the flow control device member adjusts flow resistance through the body by selectively restricting flow through the at least one variable resistance orifice.

In certain embodiments, a flow control valve is provided comprising: a valve body having; a chamber formed therein, wherein the chamber is divided by a movable piston into an inlet plenum connected to the inlet and an outlet plenum connected to the outlet, said movable piston having an orifice formed there through; an inlet connected between a proximal end of the chamber and an aspiration line by a lure fitting; a filter contained within the inlet; an outlet connected between the distal end of the chamber and the aspiration line by a lure fitting, wherein the outlet plenum has at least one variable resistance orifice connected to the outlet; wherein the movable piston is adapted to adjust flow resistance by selectively changing flow through the at least one variable resistance orifice responsive to a differential pressure between the inlet plenum and the outlet plenum. In certain aspects, the flow control valve may also contain at least one flow bypass orifice.

In certain embodiments there is provided a method of controlling flow rate through a device comprising the steps of: sensing a flow rate between an inlet and an outlet of a body; if the flow rate increases, adjusting a position of a member such that a flow resistance is increased; and if the flow rate decreases, adjusting the position of the member such that a flow resistance is decreased; whereby an increase in flow rate is countered by an increase in flow resistance and a corresponding mitigation of the increased flow rate, and a decrease in flow rate is countered by a decrease in flow resistance and a corresponding mitigation of the decreased flow rate. Such that on the whole, increasing pressure differential applied to the device inlet and outlet results in increasing flow resistance of the device proportionally to the pressure changes such the ratio of pressure divided by resistance (which is proportional to the flow rate) becomes stabilized by the action of the device.

In one embodiment there is provided a valve for controlling the flow of fluid in an aspiration tube, the valve comprising: a valve body having a chamber therein and an inlet into and an outlet from the chamber; a partition member located within the chamber between the inlet and the outlet, the partition member dividing the chamber into an inlet side and an outlet side, the partition member being movable under the influence of a difference in pressure between the two sides of the chamber; a valve seat located between the outlet side of the chamber and the outlet; a valve closure member movable with the partition member between an open position in which the valve closure member is remote from the valve seat and a closed position in which the valve closure member interacts with the valve seat, or control orifice, to either restrict or shut off the flow of fluid through the outlet; biasing means for biasing the partition member to a position in which the valve closure member is open; and a restricted flow passage between the two sides of wherein the biasing means is selected so as to provide a biasing force which is adapted to allow the partition member to move to close, or restrict the valve when the flow rate through the restricted flow passage exceeds a pre-determined flow rate. As used in this embodiment, equalization of pressure is understood to mean a return to an acceptable pressure differential or acceptable differential pressure range between the two sides of the chamber.

In another embodiment there is provided a valve for controlling the flow of fluid in an aspiration tube, the valve comprising: a valve body having a chamber therein and an inlet into and an outlet from the chamber; a partition member located within the chamber between the inlet and the outlet, the partition member dividing the chamber into an inlet side and an outlet side, the partition member being movable under the influence of a difference in pressure between the two sides of the chamber; a valve seat located between the outlet side of the chamber and the outlet; a valve closure member movable with the partition member between an open position in which the valve closure member is remote from the valve seat and a closed position in which the valve closure member interacts with the valve seat, to either restrict or shut off the flow of fluid through the outlet; biasing means for biasing the partition member to a position in which the valve closure member is open; and a restricted flow passage between the two sides of the chamber such that the pressure differential developed between the two sides of the chamber thereby created by the partition member becomes stabilized by the action of the valve when the flow rate through the restricted passage exceeds a predetermined value.

In another embodiment there is provided a device for controlling the flow of fluid in an aspiration system, the device comprising: a device body having a chamber therein and an inlet into and an outlet from the chamber; a member located within the chamber between the inlet and the outlet, the member substantially dividing, or dividing, the chamber into an inlet portion and an outlet portion, the member being movable under the influence of a difference in pressure between the two portions of the chamber between an open position and a closed position to either restrict or shut off the flow of fluid through the outlet; biasing means for biasing the member to a position in which the member is open; and a restricted flow passage between the two portions of the chamber such that the pressure differential developed between the two portions of the chamber thereby created by: the partition member becomes stabilized by the action of the member when the flow rate through the restricted passage exceeds a predetermined value.

In certain embodiments, there is provided a device for controlling the flow of fluid in an aspiration tube, the device comprising: a body having a chamber therein and an inlet into and an outlet from the chamber; a partition member located within the chamber between the inlet and the outlet, the partition member dividing the chamber into an inlet side and an outlet side, the partition member being movable under the influence of a difference in pressure between the two sides of the chamber; a stopper located between the outlet side of the chamber and the outlet; a closure member movable with the partition member between an open position in which the closure member is remote from the stopper and a closed position in which the closure member interacts with the stopper to either restrict or shut off the flow of fluid through the outlet; biasing means for biasing the partition member to a position in which the closure member is open; and the partition member has a restricted flow passage which results in a pressure differential across the partition member with flow via the restricted passage. This pressure differential opposes the biasing means and can cover a range.

In other embodiments there is provided a device for controlling the flow of fluid in an aspiration tube, the device comprising: a valve body having a chamber therein and an inlet into and an outlet from the chamber; a partition member located within the chamber between the inlet and the outlet, the partition member dividing the chamber into an inlet side and an outlet side; a valve seat located between the outlet side of the chamber and the outlet; pressure sensor means for detecting a difference in pressure between the inlet and outlet sides of the chamber: a valve closure member movable between an open position in which the valve closure member is remote from the valve seat and a closed position in which the valve closure member interacts with the valve seat to either restrict or shut off the flow of fluid through the outlet; and a restricted flow passage between the two sides of the chamber which results in a pressure differential between the two sides of the chamber, and the flow of fluid to occur through the valve between the inlet and the outlet when the valve closure member is in its open position; wherein the valve closure member is moved to an open or closed position in response to a difference in pressure detected by the pressure sensor means. In certain aspect, this pressure differential opposes the biasing means and can cover a range of pressures.

In certain embodiments, in operation the member is biased toward an open position allowing fluid to flow at acceptable flow rates, then as needed due a change in pressure differential that is not acceptable, the member moves towards a closed position further restricting the flow of fluid from the outlet until a sufficient regulation of flow rate between the inlet and outlet to the device is reached, and then the member moves back toward an open position and reaches an equilibrium where the flow rate is stabilized.

In certain embodiments, in operation the member is position to allow fluid to flow from the inlet to the outlet of the device at acceptable flow rates, as unacceptable flow rates via the device are detected the member is positioned to further restricting the flow of fluid through the device until a sufficient change in flow rate occurs and acceptable flow rates are achieved, the member is then positioned to allow fluid to flow through the device at acceptable flow rates, and the process is repeated as needed.

In certain embodiments, in operation of a device the member is position to allow fluid to flow from a first portion of the device to a second portion of the device at acceptable flow rates, as an unacceptable difference in flow rate via the device is detected at the restricted passage of the moving partition member, the member is then positioned to alter the flow of fluid through the entire device until a sufficient change in flow rate occurs and acceptable flow rates are achieved, and then the member is positioned to allow fluid to flow through the device at acceptable flow rates, and this process of regulating the acceptable flow rates is repeated as needed.

In certain embodiments, in operation a member is positioned between a first portion of a device and a second portion of the device to allow fluid to flow from a first portion of to a second portion at acceptable flow rates, when an unacceptable difference in flow rate via the partition member is detected, the member is positioned to alter the flow of fluid through the first portion and the second portion until a sufficient change in flow rate occurs and acceptable flow rates are achieved, and then the member is repositioned to allow fluid to flow through the device at acceptable flow rates, and this process is repeated as needed.

In certain aspects, the restricted flow passage is typically formed so as to be located within the valve closure member. In other aspects, the restricted flow passage is located with the member. In other aspects the restricted flow passage can be a separate structure within the device.

The ability to control flow rate and pressure stability in of considerable advantage to the safety, effectiveness and speed of the procedures. Another advantage is that eye's anterior chamber geometry can be maintained with less risk of collapse. Another advantage of certain embodiments is that the flow control device can be treated as a disposable due to the low cost to produce the device, and therefore, could be used only once, which could reduce potential contamination problems associated with non-disposable surgical equipment. A further advantage, is the flow control can be packaged with other disposables and marketed and sold by the manufacturer to doctors, clinics, and hospitals as a disposable package. This approach is attractive to doctors, clinics, and hospitals because it cuts down the chances for cross contamination between patients. Another advantage of certain embodiments is that the flow control device can be added to existing systems by placing the device in the aspiration systems of existing phacoemulsification machines thereby improving performance and patient safety. Another advantage of certain embodiments is that the procedure can be conducted at higher aspiration vacuums in Venturi machines, and higher maximum occlusion vacuums in Peristaltic machines than are typical in many known systems because excessive flow rates, which destabilize the geometry of the eye's anterior chamber, are avoided. This shortens the length of time of the procedure and the amount of time the probe must be in the patients eye. This results in less chance of damage to the eye or other side effects.

### BRIEF DESCRIPTION OF THE DRAWINGS

These and other features, aspects, and advantages of the present invention will become better understood with regard to the following description, appended claims, and accompanying drawings where:

FIG. 1 illustrates an exemplary embodiment of a peristaltic phacoemulsification system;

FIG. 2 illustrates an exemplary embodiment of a Venturi phacoemulsification system;

FIG. 3 illustrates an exemplary embodiment of a flow control device;

FIG. 4 illustrates an electrical circuit representation of the operation of an embodiment of the device;

FIG. 5 illustrates an electrical circuit representation of the operation of an embodiment of the device;

FIG. 6 illustrates another exemplary embodiment of a flow control device;

FIG. 7 illustrates another exemplary embodiment of a flow control device;

FIG. 8 illustrates another exemplary embodiment of a flow control device;

FIG. 9 illustrates another exemplary embodiment of a flow control device utilizing electro-mechanical components;

FIG. 10 illustrates the pressure conditions that may occur in the anterior chamber of the eye due to a post occlusion surge without a flow control device;

FIG. 11 illustrates the pressure conditions that may occur in the anterior chamber of the eye due to a post occlusion surge with a flow control device; and

FIG. 12 illustrates the mechanism that may lead to the collapse of the anterior chamber when the flow rate exceeds a certain value.

### DETAILED DESCRIPTION OF THE EMBODIMENTS

High outflow rates (aspiration flow), either transient or continuous, may compromise the anterior chamber's stability and geometry due to the limitation of the fluid inflow (irrigation system). These limitations relate to the flow resistances of the small caliber irrigation instruments which are required for modern small incision cataract surgery. On the other hand flow rates which are too low can cause problems with phaco needle heating as the flow rates cool the phaco-emulsification (ultrasound) crystals and the phaco needle connected to them. Also low flow rates reduce the clearance of cataract debris from the eye slowing the speed of the surgery. Therefore the flow rates during surgery should be maintained within an acceptable range to help avoid either types of problems. As discussed herein, the acceptable ranges of flow rates and the acceptable ranges of pressures can vary over quite a range depending on the set up of the system being used and the physician's operating parameters. Acceptable flow rate ranges and acceptable pressure ranges can vary also depending on whether the procedure is being carried out with a Venturi type system or a Peristaltic type system.

The flow control systems, methods, and devices disclosed herein permit better control of the pressures and flow rates used during eye surgery, for example, during phaco-emulsification cataract surgery. Specifically, some embodiments may have certain properties relevant for addressing the problem of controlling transient flow disturbances and maintaining constant flow at low vacuums during cataract surgery. Certain embodiments disclosed may be used with various Venturi-based or peristaltic based phaco machines.

An eye's anterior chamber typically contains about 0.2 ml of fluid. This can vary depending on the geometry of the particular eye being operated on. This chamber can be subject to unstable geometry conditions of too much fluid volume or too little volume during the surgery. The desirable parameters to be maintained will vary and many of the embodiments of the present disclosure may not be limited to a particular set of parameters, as long as satisfactory results are achieved with the control flow devices disclosed. However, for example, using certain embodiments, it is possible to avoid, or reduce, either large dynamic (transient) or large static (continuous) pressure variations below, for example, about 10 mmHg or above, for example, about 70 mmHg, thereby keeping the eye close to physiologic pressures during the surgery. Using certain embodiments, it is possible to maintain, or substantially maintain the eye's anterior chamber volume to not less than, for example, about one half of its physiological volume so the pressure not less than about 10 mmHg and not more than about 4 times its physiological volume at the higher pressure end of about 70 mmHg or about 80 mmHg. In certain embodiments in order to maintain the appropriate volume in the eye's anterior chamber during the procedure, it is desirable that the volume displacement of the movable member, value, or piston be kept to a low value, (e.g., less than the volume of the anterior chamber) and its response time is quick enough to neutralize flow transients by rapid flow regulation that may be induced during the procedure.

In certain embodiments the partition member, closure member, valve, piston, or member made of metal, plastic (e.g., ABS or other medically suitable plastics), silicon, or any other suitable material or combinations thereof. In certain embodiments it will be made of an acceptable medically suitable plastic.

In certain embodiments it may be important to configure the partition member, closure member and/or biasing force (collectively the “moving structures”) so that there is minimal motion. The overall compliance of the moving structure, member or member means is acceptable if the volume displacement incurred on account of the compliance is small compared to the volume of the eye's anterior chamber. Therefore the moving structure, member, or member means internal volume change is kept down to a low value. A small physical movement, dx typically 0.3 mm to 1.0 mm, depending on the diameter of the moving structures, is arranged to produce a very small physical movement, dx typically 0.3 mm to 0.5 mm, of the moving structures, is arranged to produce a very large change in Rv by occluding a small orifice. Once the critical flow rate is reached then Rv is controlled, so that the flow rate is stabilized to close to the selected value, regardless of large alterations of the vacuum at the devices outlet. In certain embodiments, it is desirable that the volume displacement of the moving structure, movable, member, valve, or piston be less then 65%, 55%, 50%, 45%, 40%, 30%, 20%, 15%, 10%, 8%, 5%, 2% or 1% of the volume of the anterior chamber. In certain embodiments, it is desirable that the volume displacement of the moving structure, movable member, valve, or piston be less then 0.5 ml, 0.4 ml, 0.3 ml, 0.2 ml, 0.18 ml, 0.16 ml, 0.15 ml, 0.13 ml, or 0.1 ml or less. In certain embodiments, it is desirable that the dx of moving structure, member, valve, or piston be within the device be between 0.1 mm to 1.5 mm, 0.3 mm to 1 mm, 0.2 mm to 0.8 mm, 0.3 mm to 0.8 mm, 0.3 mm to 0.5 mm, or 0.4 mm to 0.8 mm.

In certain embodiments, the biasing means and the partition member are separate structures, in other embodiments the biasing means and the member can be combined into the same structure. The biasing force is applied by the biasing means. Any structure, or combination of structures, that is capable of applying an appropriate biasing force may be used. The biasing means may be, for example, a spring, a plate, a rod, a piston, a membrane, diaphragm or combinations thereof and may be made of any appropriate materials. The biasing means may be disposed in a chamber that may be configured in a variety of shapes. For example, the chamber containing the biasing means (e.g., a spring) may have a conical taper on its output side. In this example, the spring may possess an initial compression force (also referred to as the biasing force). In still further embodiments, the biasing force is provided by both the partition member and the biasing means. For example, the partition member may be a membrane or diaphragm having a certain capacity to apply a biasing force to the member which is completed by a biasing means in the form of a spring. Additionally, the partition member may be a spring and piston combination with an additional spring. Where the biasing force is applied by the biasing means only, or by the biasing means acting together with the partition member, the biasing means and/or partition member may be adapted to engage with each other directly, or via a further member, to apply a biasing force to the partition member that holds the member to a position in which the valve closure member is open. The desired biasing force can vary depending on a number of other related structural, flow resistance, and fluid flow factors. The spring force can be predetermined or modified to meet the needs of a particular device. In certain embodiments the biasing force will have between 2 grams to 50 grams, 5 grams to 40 grams, 2 grams to 40 grams, 10 grams to 30 grams, or 5 grams to 15 grams of spring force. In other embodiments the biasing force will be predetermined at between 2 grams to 50 grams, 5 grams to 40 grams, 2 grams to 40 grams, 10 grams to 30 grams, or 5 grams to 15 grams of spring force. In other embodiments the biasing force will be predetermined at 2, 5, 8, 10, 12, 14, 15, 20, 25, 30, 35, 40, 45 or 50 grams of spring force. For example, when the orifice 17 has a flow resistance of 3×109 where the units of resistance are metric: Newton. seconds/meters to the power of 5 and the spring has 12 grams of spring force, the piston will dynamically adjust to regulate the flow rate around 30 ml/min.

In certain embodiments, the chamber, or chambers, inside the device may be shaped in a variety of shapes and geometries. In designing the geometry or shape of the chamber the design may take into consideration the ease with which fluid can move through the chamber interior.

In certain embodiments, the device further includes one or more debris filters to prevent and/or minimize tissue debris and/or trapped air bubbles from interfering with the operation of the member. The location of the filter can be in outside the device in some embodiments and will typically be located before the member so as to remove and/or minimize tissue debris from interfering with the workings of the member. In certain embodiments, the chamber further includes one or more debris filters located in the inlet side of the chamber to prevent and/or minimize tissue debris and/or trapped air bubbles from interfering with the operation of the device. For example, in some aspects a fish net filter may be employed. In other embodiments, the filter can be in a longer tubing section so as to move it away from the probe to minimize entanglements. The filter may be made of any acceptable material, for example, from a synthetic material, a plastic material, or a metallic material or combinations thereof. The fliter mesh is such that cataract particles which could clog the fluid flow apertures in the device are filtered and caught in the net or mesh. Therefore, in certain embodiments, the texture or gaps in the mesh/filter have a smaller size than the smallest orifice in the device and are therefore typically less than about 0.1 or less than about 0.2 mm in size.

The pressure sensor means may operate on a force transducer or piezo resistance principle, although any pressure sensing mechanisms may be employed. In the electronic version which senses the pressure, any strain gauge, piezo-resistive, or any sensor using electrical capacitance or electrical inductance or combinations thereof, or any pressure transducer which converts pressure changes to an electrical signal may be employed.

In certain embodiments, in operation the member is in an open position allowing fluid to flow, and then as needed the member moves towards a closed position further restricting the flow of fluid from the outlet and between the portions of the chamber enabling the control of and the stabilization of flow. As used herein, open position and closed position are defined to include, substantially open or closed, partially opened and closed, and/or reasonable gradations. The use of open, in certain embodiments herein can mean fully opened, substantially opened, partially opened, and/or reasonable gradations of open or a member that is biased toward an open position. The use of closed in certain embodiments herein can mean fully closed, substantially closed, partially closed, and/or reasonable gradations of closed or a member that is biased toward a closed position. The use equalization of pressure in certain embodiments herein can mean the pressure difference between two chambers is stabilizes to a fixed numerical value greater than a zero value.

In certain embodiments, the stabilization of a difference in pressure can be defined as the sum of spring pressure (Ps) plus pressure in the second portion of the chamber (P2) being equal to being equal to the pressure in first portion of the chamber (P1). In some embodiments, stabilization of pressure can be defined as the movement of Ps plus P2 towards being equal with P1. A bypass flow may be provided to prevent total flow occlusion and limit the maximum overall flow resistance that the device can acquire

In certain embodiment, in operation the member may be in an open position allowing fluid to flow, and then as needed the member moves towards a closed position further restricting the flow of fluid from the outlet and between the portions of the chamber enabling the stabilization of flow, or the movement towards stabilization of the flow, via the device. In certain embodiments, the stabilization of flow throughout the device results from an equalization of forces where the pressure drop across a piston results in a force on the piston which is exactly equal to the force in the spring, and the piston assumes a physical position to control the flow resistance such that the flow rate through the entire device is stabilized.

Embodiments of the present invention may have a male/female lure, like a short extension, configured so that they can be attached in the aspiration tubing line directly on the probe where the aspiration line would normally attach.

In certain embodiments, the inlet may be configured in a variety of geometries. For example, in certain embodiments, the inlet may be a bi-conical shape formed by a member or partition member (e.g., a piston) and the valve body. In these embodiments, the piston motion stops may be part of the valve body. Such a configuration of a bi conical chamber may advantageously help bleed air out of the system.

In certain embodiments, the control flow device is provided as a disposable unit. In certain embodiments the control flow device may be formed integrally with an aspiration tube and/or other components used in line in the aspiration of tissues and fluids, such as a tissue/fluid collection bag. In some embodiments, the control flow device will be provided as a disposable system that includes, tubing, bags, fittings, the vacuum sensor interface, directions on how to use the device in the system, or any combination of the above. In some embodiments some parts of these systems will not have to be disposable and may be reused.

Acceptable and unacceptable flow rates and vacuums may depend on a number of characteristics of the system, the patient, and the procedure. Therefore, these parameters can vary significantly with certain embodiments of the present disclosure For example, flow rate generally varies proportionately with the bottle height and vacuum (generated by either the Venturi system or the peristaltic pump), and inversely with the total flow resistance (irrigation and aspiration system). For example, provided the outflow rate from the eye, which includes the aspiration flow rate, and any leakage flow rate, does not exceed 40 ml/min, then the pressure loss along the irrigation pathway would be only about 32 mmHg (with typical irrigation apparatus). Accordingly, flows rates and vacuums can vary significantly using the embodiments disclosed.

In certain embodiments, acceptable flow rates can vary between 5 ml/min to 40 ml/min, 10 ml/min to 40 ml/min, 10 ml/min to 38 ml/min, 10 ml/min to 35 ml/min, 15 ml/min to 35 ml/min, or 20 ml/min to 35 ml/min. To achieve acceptable flow rates, any suitable combination of bottle height, vacuum, and flow resistance could be utilized. For example, in certain embodiments, the bottle height can vary between 30 cm to 65 cm, 65 cm to 80 cm, 80 cm to 120 cm, or 120 cm to 200 cm. In certain embodiments, vacuum can vary between 5 mmHg to 150 mmHg, 5 mmHg to 140 mmHg, 5 mmHg to 120 mmHg, 10 mmHg to 130 mmHg, 10 mmHg to 120 mmHg, 40 mmHg to 200 mmHg, or 15 mmHg to 100 mmHg. Higher values of vacuum may be employed if the venture set disposables have a higher than usual flow resistance, or if flow restrictors, such as narrow apertures, or small internal diameter phaco needles are employed. In certain embodiments, the majority of the total flow resistance is typically in the aspiration system, e.g. approximately 20% is in the irrigation pathway and approximately 80% in the aspiration pathway.

In a Venturi machine, the flow rate depends on the bottle height, the machines vacuum setting, and the overall flow resistance of the entire fluidic system. For example, when the vacuum is at modest values (e.g., 200 mmHg) with a typical bottle height of 70 cm, the flow resistance may be such that the flow rate will approach 60 ml/min (unbeknownst to the surgeon), which is undesirably high. Such a high flow rate could reduce the anterior chamber pressure to a dangerously low level causing the chamber to collapse.

In many systems (including systems using Venturi or peristaltic machines), to maintain anterior chamber pressure stability, embodiments of the present invention may control the flow rate for both average values, e.g., in Venturi machines (e.g., not to exceed 20. 25 30 or 40 ml/min in certain systems or not to exceed 50 ml/min in certain systems) and similar peak transient values or higher which occur in Peristaltic machines. In these applications, there may be a pressure loss along the irrigation pathway due to flow resistance. This pressure loss can occur with typical caliber irrigation instruments and, especially, in the narrow caliber irrigation instruments used in small incision cataract surgery. For example, when the flow rate exceeds 60 ml/min or 65 ml/min, 50 mmHg pressure can be lost (dissipated) by the irrigation flow resistance, and if he bottle is providing 50 mmHg (70 cm), all the pressure is dissipated and the eye's anterior chamber pressure falls to zero.

In Venturi machines, embodiments of the present invention solve, or reduce, this problem by limiting the flow rate when it exceeds, for example, 30 ml/min and stabilizing the flow rate to that value. In this manner the venture machine's vacuum can be increased and bottle adjusted without compromise to the anterior chamber pressure under constant unoccluded flow situations.

Peristaltic machines operate such that the average unoccluded flow rate is well controlled by the peristaltic pump in the machine up to a value of, for example, 30 ml/min. In these circumstances a typical Peristaltic pump machine generates a secondary vacuum, of around 60 mmHg, which is a low value compared to the typical vacuums of 100 to 120 mmHg used in a venture machine. However, flow instabilities can still occur even with the low unoccluded vacuum levels and controlled un-occluded flow rates. For example, this may be caused by stored energy in the elastic structures (e.g., aspiration tubing, pump tubing and vacuum sensors). In peristaltic systems the flow peaks can be a range of values at occlusion break (when the post occlusion surge appears) and are generally proportional to the maximum allowable occlusion vacuum level set on the machine, by the surgeon, which is the value typically in the aspiration system prior to occlusion break. Typical values used by surgeons currently are 250 to 350 mmHg, and this vacuum only occurs in the absence of significant flow because the flow is occluded to allow the vacuum to be generated by the pump continually removing fluid from the elastic aspiration system.

Higher occlusion vacuums over 300 mmHg result in significant post occlusion surge instability in the peristaltic machine. For example, with a maximum vacuum of 500 mmHg, typically the peak flow rate immediately (e.g., around 70 milliseconds) after occlusion break can be 100 ml/min. This may collapse the eye's anterior chamber because of the pressure losses with flow due to the flow resistance in the irrigation pathway.

In peristaltic machine applications, embodiments of the present invention may respond quickly enough (e.g., less than 70 milliseconds) to minimize the flow rate and prevent collapse of the anterior chamber.

In many systems (including systems using peristaltic or Venturi machines) unstable pressures during eye surgery are generally not desirable and need to be minimized using embodiments disclosed herein. Unstable pressures and flow rates of fluids into an eye's anterior chamber can alter anterior chamber's geometry. This instability can result in the collapsing of the eye's anterior chamber, and this could damage the eye's delicate tissues. Complications include lens capsule rupture, iris damage and corneal damage. Capsular rupture predisposes to other complications such as glaucoma, macula oedema and retinal detachment. By unstable pressure we typically mean large fluctuations in pressure between the bottle pressure, for example, a fluctuation of 50 mmHg with a 70 cm bottle height and a low pressure of zero or a negative value. Stable pressures can be defined as the eyes physiological pressure of 10 mmHg to 21 mmHg, and including higher pressures up to 80 mm Hg provided by the bottle. However an eye's chamber may collapse with a positive pressure value of 10 mmHg or more if there is external pressure on the globe from the orbital tissues, anesthetic fluid pressure from the eye lid speculum, or contraction of extra ocular muscles. The internal eye pressure also drops with leaky surgical wounds because this increases the irrigation flow rate and therefore the irrigation pressure losses due to irrigation resistance. In practice a flow rate of 30 ml/min results in a pressure loss of about 25 mmHg along the irrigation pathway resistances. Therefore with a bottle height of 70 cm (approximately 50 mmHg) there will be a 25 mmHg pressure fluctuation at least when the flow stops and starts with occlusion make and break. This would be acceptable, as with continuous flow, the anterior chamber pressure would be 25 mmHg, which would allow for any external pressure on the globe and give a well formed chamber, and the chamber may deepen slightly as the pressure fluctuates toward the 50 mmHg with occlusion.

In many systems (including systems using peristaltic or Venturi machines), with all the variables involved, exact acceptable ranges may vary using embodiments disclosed herein. For example a 25 mmHg fluctuation could be acceptable with a 50 mmHg (70 cm) bottle height and no external globe pressure, because the eye pressure would not dip below 25 mmHg. However with a 40 cm bottle height (30 mmHg) and 5 mmHg to 10 mmHg pressure on the outer globe, the chamber could collapse with a 25 mmHg pressure fluctuation caused by the usual 30 ml/min flow rate. Any wound leakage would make the situation worse and the numbers different.

In many systems (including systems using peristaltic or Venturi machines), if the flow rates are limited to the range of 20 ml/min to 40 ml/min, then the pressure fluctuations associated with this (due to irrigation pathway limitations) are in the range of 15 mmHg to 35 mmHg. Then provided the bottle pressure is at least 50 mmHg (approximately 70 cm irrigation bottle height), the lowest pressure, excluding wound leak the eye will experience is 15 mmHg. If there is pressure on the globe's outer wall this effectively subtracts from this value, increasing the risk of chamber collapse. Wound leakage also adds to the anterior chambers pressure loss. The bottle can be increased in height to, for example, 1 m. This provides approximately 73 mmHg pressure, however in the absence of any flow this pressure may result in a very deep and difficult to view anterior chamber.

In many systems (including systems using peristaltic or Venturi machines), the ranges of pressure loss, in the eye, due to flow rate, depend on he particular set of irrigation instruments and their flow resistances. In general, a flow rate of 25 ml/min to 30 ml/min may be acceptable with conventional irrigation sets and typical bottle heights of 70 cm to 80 cm.

In many systems (including systems using peristaltic or Venturi machines, if the flow rate is too high the anterior chamber can collapse. Typically a flow rate of 65 ml/min may collapse the anterior chamber with a standard set of disposables and 70 cm irrigating bottle height. The flow rate that will cause stability problems will vary depending on the set of disposables (such as tubing and irrigation instruments used). Phaco machines that use a Venturi principle for creating a vacuum are well known for having problems because the fluid flow rate is often not well controlled. This is because these machines depend on the applied vacuum level and bottle height to control flow rate which may be variable during the surgery. Any amount of irrigation flow rate, or leakage flow rate results in a reduction of anterior eye pressure due to the resistive pressure losses along the irrigation fluid flow pathway. Using certain embodiments disclosed, a well controlled flow rate will range from of 20 ml/min to 40 ml/min depending on bottle height, particular eye, wound leak orbital pressure etc, and preferably in the order of 25 ml/min to 30 ml/min. In these circumstance you would prefer that the flow rate not be less than 15 ml/min to avoid phaco needle heating, and not greater than 45 ml/min to avoid too much pressure loss in the eye.

In certain embodiments the device disclosed will have a body connected to tubing through which fluid flows into an inlet of the body of the device and out an outlet on the body of the device. The device may have a chamber containing a valve closure member movable with a partition member between an open position and a closed position to control the flow of fluid through the device. The valve closure member and partition member may divide the chamber into an inlet portion (inlet plenum), and outlet portion (outlet plenum).

In certain embodiments, a restricted flow passage is provided on the partition member with the valve closure member being provided on the end of the restricted flow passage that opens into the outlet side of the chamber. In these embodiments, with movement of the partition member, the valve closure member is brought adjacent to the valve seat to restrict or otherwise shut off the flow of fluid through the restricted flow passage.

In other embodiments, the valve closure member is not provided on the restricted flow passage. In these embodiments, the restricted flow passage may or may not be provided on the partition member.

In certain aspects, the restricted flow passage is typically formed so as to be located within the valve closure member.

In certain embodiments the partition member of the device may be configured to bias itself to a position in which the valve closure member is open. In other words, the biasing means and the partition member may be one and the same thing. For example, the partition member may be a membrane or diaphragm that is configured to apply a biasing force that holds the membrane or diaphragm to a position in which the valve closure member is open.

In other embodiments, the biasing means and the partition member are separate elements. In these embodiments, the partition member is not configured to apply a biasing force. The biasing force is applied by the biasing means. For example, the partition Member may be a piston, a plate, and the biasing means a return spring.

In still further embodiments, the biasing force is provided by both the partition member and the biasing means. For example, the partition member may be a membrane or diaphragm having a certain capacity to apply a biasing force to the member which is completed by a biasing means in the form of a spring. Additionally, the partition member may be a spring and piston combination with an additional spring.

Where the biasing force is applied by the biasing means only, or by the biasing means acting together with the partition member, the biasing means and/or partition member may be adapted to engage with each other directly, or via a further member, to apply a biasing force to the partition member that holds the member to a position in which the valve closure member is open.

In certain embodiments it may be important to configure the partition member, valve closure member and biasing force (collectively the “moving structures”) so that there is minimal motion. Otherwise the device itself could add significant compliance (i.e., have a significant volume displacement, compared to the eye, over its working range) to the aspiration system and induce secondary problems (e.g., increased post occlusion surge). This is one reason why a spring tension return force (otherwise referred to as a “biasing force”) may be included, compressing the moving structures to a “stopper” prior to any dynamic control activity of the device, or any motion dx. If this were not the case, then the motion of the moving structures could be approximately 6 to 10 times greater than without a biasing force. In certain fluid control applications, this motion would not be very important. However, in phaco-emulsification fluid management systems, it may be desirable to minimize aspiration system compliance to the extent possible because increasing this compliance could increase the post occlusion surge magnitude as explained above.

In these embodiments, the flow of fluid through the valve creates a pressure in the inlet and outlet sides of the chamber that is detected by the pressure sensor means.

The pressure sensor means may operate on a force transducer or piezo resistance principle, although any pressure sensing mechanisms may be employed.

In certain embodiments, the pressure sensor means may be programmable to cause the valve closure member to move into an open or closed position in response to a pre-defined pressure differential detected by the pressure sensor means. In other embodiments, the pressure sensor means may be programmable to cause the closure member to move into or towards an open or closed position in response to a defined pressure differential detected by the pressure sensor means. In other embodiments, the pressure sensor means may be programmable to cause the closure member to open, or partially open, as well as close, or partially close in response to the pressure differential detected by the pressure sensor.

In one embodiment, the flow release passage is provided in the valve seat. In certain embodiments, the valve further includes a stopper member against which the partition member is located when the biasing force is applied to the partition member.

In certain embodiments, the device further includes one or more debris filters to prevent and/or minimize tissue debris and/or trapped air bubbles from interfering with the operation of the member. In certain embodiments, the chamber further includes one or more debris filters located in the inlet side of the chamber to prevent and/or minimize tissue debris and/or trapped air bubbles from interfering with the operation of the member. For example, in some aspects a fish net filter may be employed. In other embodiments, the filter can be in a longer tubing section so as to move it away from the probe to minimize entanglements. The fliter mesh is such that cataract particles which could clog the fluid flow apertures in the device are filtered and caught in the net or mesh. Therefore, in certain embodiments, the texture or gaps in the mesh/filter have a smaller size than the smallest orifice in the device and are therefore typically less than 0.1 or less than 0.2 mm in size.

The inlet of the valve may be adapted for connection to an aspiration tube. The aspiration tube may then be connected directly to a surgical instrument for use in an ophthalmic or other clinical procedure, such as a phaco emulsification probe.

The outlet of the valve may be adapted for connection to an aspiration tube. The tube may be connected to a pump for applying a vacuum, such as a Venturi mechanism, or a peristaltic pump.

In certain embodiments, the valve is provided as a disposable unit. In these embodiments, the valve may be formed integrally with an aspiration tube and/or other components used in line in the aspiration of tissues and fluids, such as a tissue/fluid collection bag.

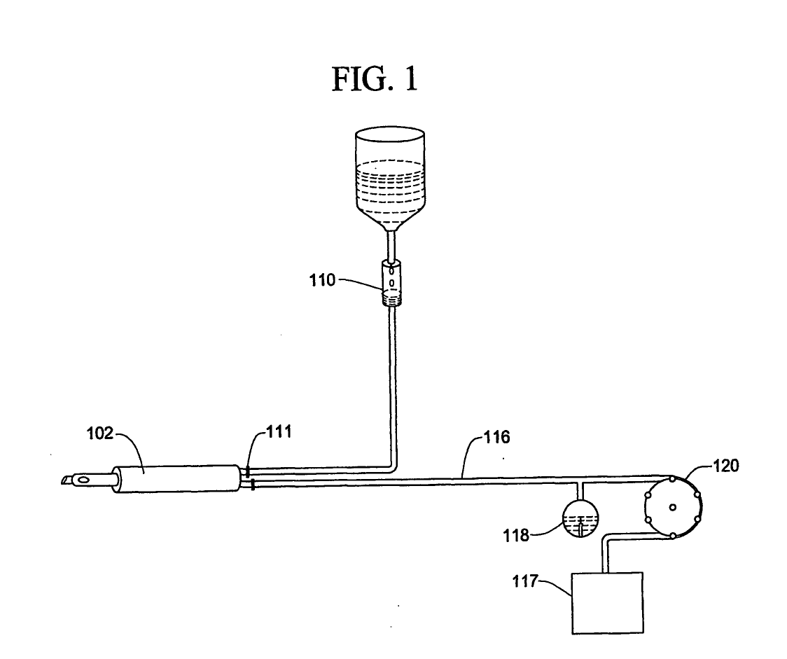

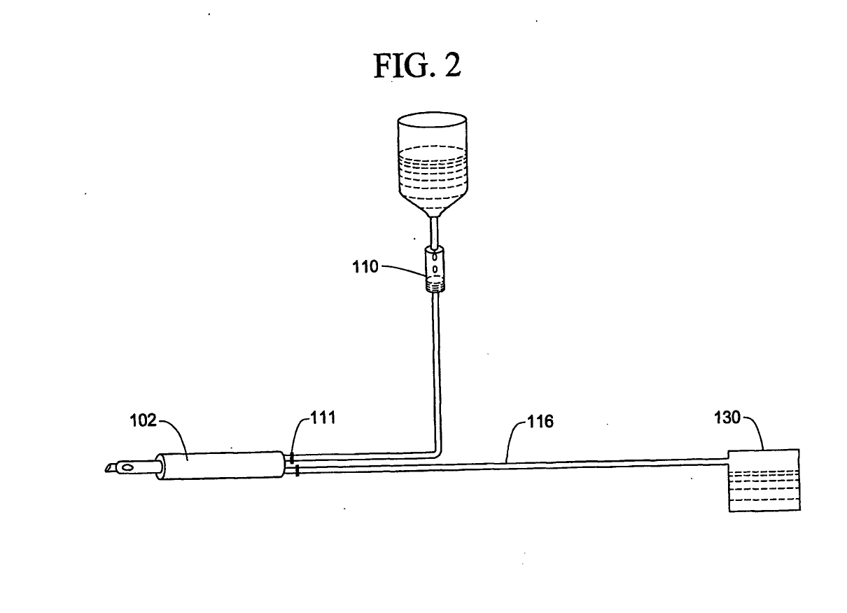

FIGS. 1 and 2 illustrate exemplary irrigation/aspiration systems. FIG. 1 illustrates a peristaltic system and FIG. 2 illustrates a Venturi system. The system comprises a number of pieces. A surgeon may utilize the handpiece or probe 102 for surgical procedures. A surgical console (not shown) controls the operation of the pumps and hand piece and provides a user display. A cylindrical chip (not shown) for fragmentation with an aspiration hole is attached to the tip of the handpiece 102. The chip is subjected to ultrasonic vibrations to perform fragmentation and emulsification of nucleus of a crystalline lens.

An irrigation bottle 110 contains an irrigation liquid such as a saline which is supplied to a patient's eye. An irrigation tube 111 leads the irrigation liquid to the eye via the handpiece 102. A pole (not shown) hangs the bottle 110, and is movable up and down. The bottle 110 may thereby change its height. The bottle 110 is arranged at such a height as to keep a pressure inside the eye properly.

One end of the irrigation tube 111 is connected with the bottle 110, and the other end is connected with the handpiece 102. The handpiece 102 may be changed to any of various kinds of handpieces including that for irrigation/aspiration according to a step in surgery, a method of surgery or the like, and the changed handpiece is connected and may be replaced with another before being used.

A flexible aspiration tube 116 is used for discharging tissue such as nucleus subjected to fragmentation and emulsification together with the irrigation liquid aspirated though the aspiration hole of the chip out of the body. In FIG. 1, in a rear direction midway along the aspiration tube 116, a peristaltic aspiration pump 120 is provided in order to generate aspiration pressure in the aspiration tube 116. A vacuum sensor 118 May also be provided in the aspiration tube 116 to provide an indication of vacuum. The aspirated liquid with the tissue is discharged and flushed into a drainage bag 117. In FIG. 2, a Venturi device cartridge/cassette 130 is be used for generating aspiration pressure.

In some embodiments directed to peristaltic systems, the flow control device may fit in the aspiration tubing that joins the probe to the vacuum sensor/peristaltic pump area in the machine. In some embodiments, the device may attach near the probe end of the setup, as a small extension to the tube. If attached further away from the probe, it may not function as well because it may not be able to deal with the stored energy in the aspiration tube and the resultant surge flow. In embodiments directed to Venturi machines the aspiration tubing may connect the probe to the air filled cassette (e.g., a small plastic box) and the device could be connected at the tube end.

In certain embodiments, the valve could be constructed of a variety of materials. For example, the valve could be constructed of flexible materials such as, for example, rubber or silicon. In still other embodiments, the whole assembly could be formed of rigid materials. In some embodiments, the assembly could be reduced to about 50 to 60 mm in length if desired.

### Examples

When a pressure gradient is applied across a fluid carrying object, such as a pipe, fluid is driven or transported from the area of higher pressure to the area of lower pressure. Layers of fluid in the tube adopt different velocities being higher in the centre and slower towards the walls. There are frictional forces between the layers of fluid and these relate to the viscosity of the fluid. There is also heat dissipation and energy loss. This is known as the resistance to flow. The energy loss is manifest as pressure loss along the flow pathway. When real fluid passes through small holes, tubes and apertures in a fluid flow pathway pressure is also lost as the fluid enters the entrance to the holes. The pressure losses given by P(loss)=Flow rate×Resistance to flow. For example, using a hole in a piston, with a resistance of, for example, 3×10 to the 9 and a flow rate of 5×10 to the minus 7, the pressure loss across the hole is 1500 Newtons per square meter or 11.2 mmHg.

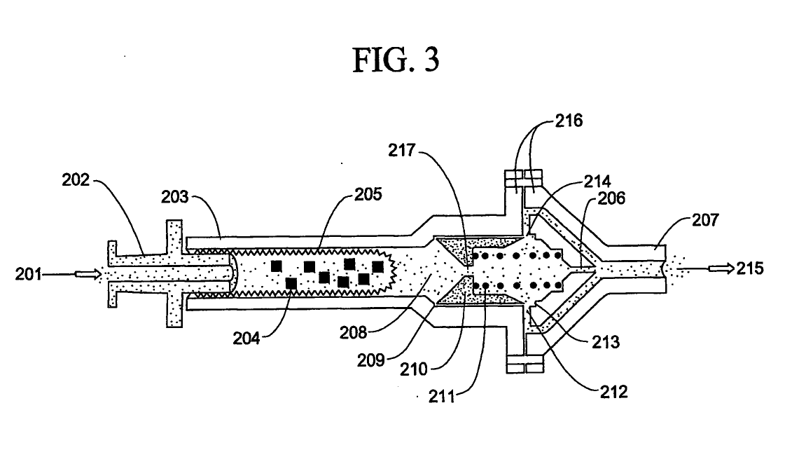

FIG. 3 illustrates an embodiment of the present invention to control flow rate. As shown, this embodiment comprises a valve body 216 having an inlet 201 and an outlet 215. The inlet comprises a female lure type fitting restrictive 202 attached to a tubing section 203. The tubing section may also contain a fishnet type or gauze filter 205. The inlet tubing section 203 is attached to a bi-conical input chamber 208 containing a piston 210. The piston may be pressed by a spring 211 (i.e. biased) against a piston return stop 209 formed into the valve body. The piston 210 contains a flow passageway leading to an orifice 217. On the outlet side of the piston, the valve body comprises a conical outlet chamber that may be attached to variable resistance flow passageways (Rv1) 212 and (Rv2) 214 and a flow bypass passageway (Rb) 206. These passageways are connected to the outlet 215, that may be configured as a male lure fitting. In some embodiments, there may be two or more of these variable resistance flow passageways to balance the pressure perpendicularly to the piston's motion. In alternative embodiments, there may be only a single variable resistance flow passageway.

As fluid flows through the orifice 217, there is a pressure loss and a corresponding pressure gradient developed across the piston 210. This pressure gradient applies a force to the piston that pushes against the force of the spring 211. When the pressure gradient force (the pressure gradient multiplied by the piston's surface area) exceeds the spring force, the piston 210 moves in a direction to occlude the variable resistance flow passageways (Rv1) 212 and (Rv2) 214. As the Rv passageways are occluded, the fluid has a smaller volume to flow through, thereby increasing the flow resistance and reducing the flow rate. The flow rate drops to a value that stabilizes the pressure across the piston 210 to a fixed value. For example if the flow rate is 30 ml/min or 5×10-7 cubic meters per second, and the orifice's flow resistance constant is 3×109, then the pressure developed across the piston is 1500 Newtons/square meter (11.2 mmHg). If the piston is 10 mm diameter then, the piston's surface area is 7.85×10-5 square meters, and the force on the piston is therefore 7.85×10-5*1500, or 0.12 Newtons (equivalent to 12 grams). Therefore, when the orifice 217 has a flow resistance constant 3×109 and the spring has 12 grams of spring force, the piston will dynamically adjust to regulate the flow rate around 30 ml/min. In a typical application of this embodiment, the initial force may be approximately 0.75 Newtons, but may be between approximately 0.01 and 5 Newtons or any other suitable value. Also, a spring constant may be on the order of 0.5 N/mm, but may be between approximately 0.01 and 5 N/mm or any other suitable value.

The variable resistance flow passageways (Rv1) 212 and (Rv2) 214 function as described above to govern flow between the inlet and the outlet. As the piston 210 is compressed against spring pressure 211 by fluid flow from the inlet to the outlet, the piston may be forced toward the outlet, covering the holes for the variable resistance flow passageways (Rv1) 212 and (Rv2) 214, thereby reducing flow through these passageways. As the piston is pushed further toward the outlet, it may be forced into the piston advance stop 213 also formed into the valve body 216. At this point, the holes for the variable resistance flow passageways Rv1 212 and Rv2 214 may be fully covered by the piston 210, thereby fully restricting flow through these passageways. In this configuration, the fluid will flow solely, primarily, or substantially through the flow bypass or release passageway (Rb) 206.

One aspect of this embodiment is that the volume displacement of the piston may be small compared to the volume of the anterior chamber of the eye (approximately 0.2 mL). In typical commercial flow regulator valves, the volume displacement is less important as it will cause no difficulties at the start of valve action. However, in phaco fluidics applications, a small piston volume displacement may be desirable. In these applications, if the piston volume displacement is relatively large, then during a fluid flow transient such as a post occlusion surge, the anterior chamber could empty out and collapse due to the piston action. Therefore it may be advantageous to minimize the piston motion dx.

The relationship between the piston diameter and the piston motion can be adjusted in any suitable range. For example, a 10 mm diameter piston and a piston motion of 0.3 mm would cause a volume displacement of 0.023 mL (i.e. around 10% of the eye's anterior chamber volume which is acceptable). Similarly, a 7 mm diameter piston with a piston motion of 0.6 mm will also generate a volume displacement of 0.023 mL. Therefore, a smaller piston can have a larger piston motion for the same volume displacement over the working range of the valve. The diameter of the piston may be any suitable value, for example it may be approximately 7 mm, and typically may be between 5 mm and 100 mm. The length of the piston may be any suitable value, for example it may be approximately 10 mm, and typically may be between 5 mm and 100 mm.

This embodiment may have several advantages. The bi conical chamber of this embodiment may advantageously help bleed air out of the system, and the filter arrangement may result in minimal trapped bubbles. The male/female lure may act like a short extension, so that the valve can fit in the aspiration tubing line directly on the probe where the aspiration line would normally push on. The tubing section 203 can be longer and flexible between the female lure fitting and the tubing section, or the filter 205 can be in a longer tubing section so as to move it away from the probe and avoid it getting in the way. The valve could also be rigid as this could reduce the size to for example, 50 to 60 mm long if desired.

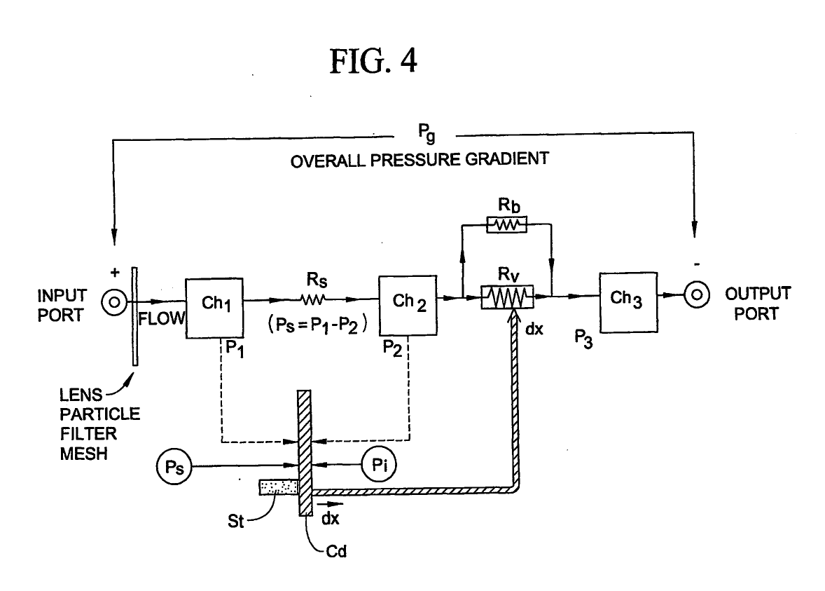

Turning to FIG. 4, there is shown a circuit diagram representation of the operation of a flow control device or valve according to an exemplary embodiment. The valve of this exemplary embodiment has certain properties relevant for addressing the problem of controlling transient flow disturbances and maintaining constant flow at low vacuums during cataract surgery as follows:

a sensing resistance to fluid flow (Rs) which generates a sensing pressure Ps, in proportion to the flow via Rs;

a compliant structure Cd. Ps is applied to Cd. Cd is also referred to herein as the partition member 304. Cd can move physically in response to Ps, some small distance dx. Cd may include a spring or membrane or spring/membrane or spring and piston combination. Cd can be a metal, plastic membrane or piston with or without an additional spring. As discussed herein, where the partition member 304 and biasing means 309 are separately formed, the biasing means 309 may be a spring or the like.

a variable resistance (Rv) formed between the chamber Ch2, and another chamber Ch3. As discussed above, Rv is provided when the valve closure means 308 shuts-off or interferes with fluid flow through the outlet by interaction with the valve seat, 307. Further, in certain embodiments, Rv is provided in the outlet side of the chamber, represented in FIG. 4 as Ch2 and Ch3. Rv is created by the moving part (in this case the partition member 304) which carries Rs, approaching the surface of the boundary of a valve surface within Ch3 (the “valve surface” being described as the “valve seat” 307 in the embodiments described above). This creates the variable orifice in which changes in geometry (and flow resistance) according to the movement (dx) of the moving parts carrying Rs. Rvi is the initial value of Rv prior to any control by dx. The maximum value that Rv can reach can be limited to that set by a bypass resistance Rb (referred to as the “flow release passage” 313), shunting Rv, or a mechanical limit to the motion dx, before the aperture creating Rv fully closes.

“control offset” is a “pressure setting” within the valve, which represents an initial pressure acting on (or part of) Cd and represented by some initial pressure Pi. Typically it can be the compliant structure itself biased toward a “stopper” 314, or a separate compression spring (referred to above as a biasing means 309) with initial compression force (referred to above as a biasing force). This initial force has to be overcome by the pressure gradient Ps, generated across Rs prior to any motion dx. Ps is generated by the flow via Rs, so the flow via Rs has to reach an initial critical value Fc, (Fc Ps/Rs), prior to the moving structures (carrying Rs) being physically able move at all. In certain embodiments it is important to configure this arrangement so that there is minimal motion of the moving structures to execute control over Rv. Otherwise the valve itself would add significant compliance to the aspiration system and induce secondary problems. This is why there is a spring tension return force (otherwise referred to as a “biasing force”), compressing the moving structure to a “stopper” 314 prior to any dynamic control activity of the valve, or any motion dx. If this were not the case, then the motion of the moving parts would be on the order of 6 to 10 times greater without this feature. In many fluid control applications this would not matter at all, but in Phaco-emulsification fluidic applications, it may be important to keep the aspiration system compliance Cm as low as possible, as increasing this compliance Cm also increases the post occlusion surge magnitude as explained above. The “Control Offset” in this instance performs two functions: it sets the flow rate at which the valve starts to adjust Rv, and it provides Pi, the initial pressure that must be overcome prior to any motion of the moving parts. This significantly reduces the overall compliance of the valve because internal movements dx, of the moving parts is then limited to 0.3 mm to 0.6 mm over the full range of vacuum, eg 0 to 600 mmHg (or pressure gradient) applied to the valve. In other embodiments, the overall compliance of the closure member or means is acceptable if the volume displacement incurred on account of the compliance is small compared to the volume of the eye's anterior chamber, or small compared with 0.2 ml. Therefore the valve's internal volume change over this pressure range is kept down to a low value 0.15 ml. A small physical movement, dx typically 0.3 to 0.5 mm, of the moving structures, is arranged to produce a very large change in Rv by occluding a small orifice. Once the critical flow rate, for example 30 ml/min (or any selected value 15 to 45 ml/min) is reached then Rv is controlled, so that the flow rate is stabilized to close to the selected value, regardless of large alterations of the vacuum at the valves outlet. In other words, as the pressure gradient across the device varies, the flow rate, above a threshold value, remains constant.

another property important in certain embodiments is that of a certain value Hysteresis, such that the valve can respond fast enough (e.g., in less than 70 milliseconds) to rapid changes in either applied vacuum or flow rate which occur during the post occlusion surge. In certain embodiments, the appropriate value may be selected by selecting the mass and geometry of the moving parts and the compliant structures, so as to suit the “fluidic transients”, “constant flow” situations and “occlusion of flow situations” which occur during phaco-emulsification cataract surgery. So therefore plastic lightweight components for the moving parts are suitable, but metal parts of low density and mass may also be usable.

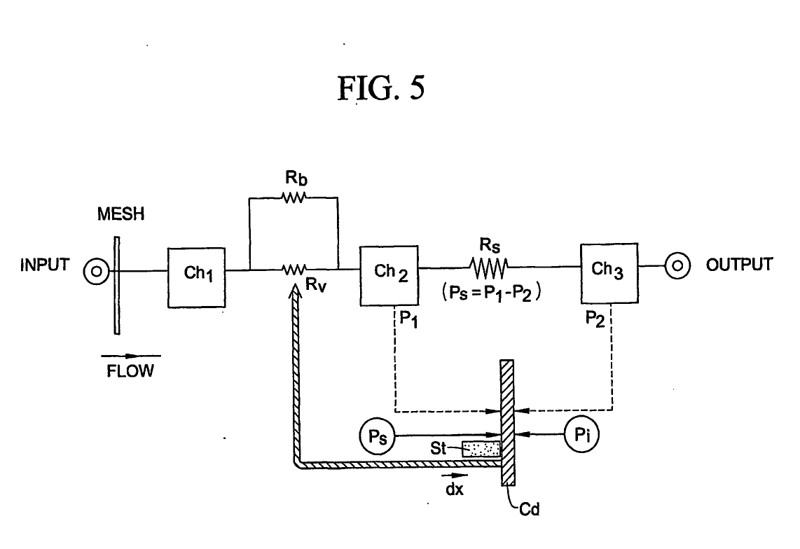

FIG. 5 shows an alternative arrangement of a feedback system similar to the feedforward system of the embodiment shown in FIG. 4.

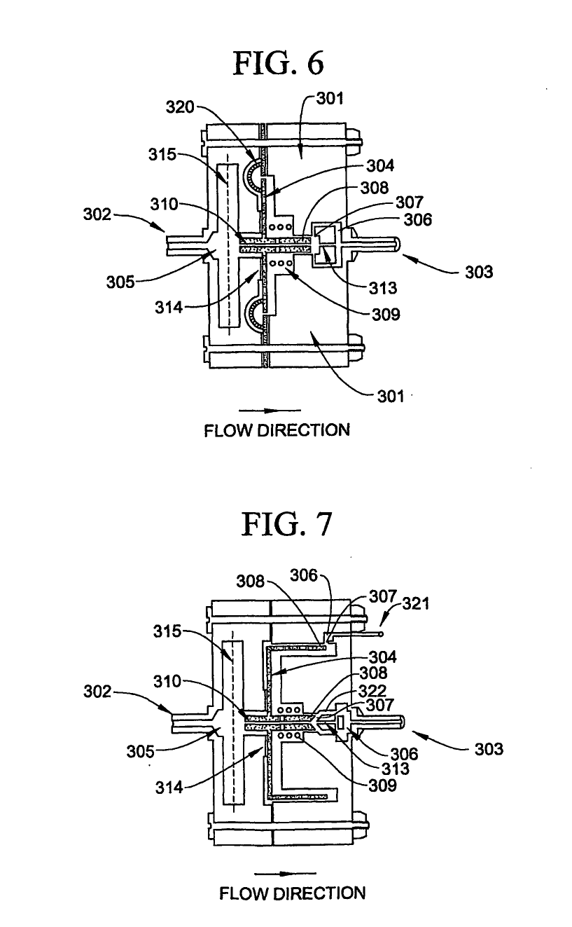

As shown in FIGS. 6 to 8, the valve may include a valve body 301 having a chamber therein and an inlet 302 into and an outlet 303 from the chamber. A partition member 304 located within the chamber between the inlet and the outlet divides the chamber into an inlet side 305 and an outlet side 306. The partition member is movable under the influence of a difference in pressure between the two sides of the chamber. The chamber may include In the inlet side, a debris filter 315. A valve seat 307 is located between the outlet side of the chamber and the outlet 303. A valve closure member 308 movable with the partition member 304 between an open position in which the valve closure member is remote from the valve seat 307 and a closed position in which the valve closure member interacts with the valve seat to either restrict or shut off the flow of fluid through the outlet. There is also a biasing means 309 which biases the partition member 304 to a position in which the valve closure member is open. That position may be demarcated by stopper 314. A restricted flow passage 310 is located between the two sides of the chamber enabling the equalization of pressure between the two sides of the chamber, and the flow of fluid to occur through the valve between the inlet and the outlet when the valve closure member 308 is in its open position. The restricted flow passage 310 may be any suitable size, for example 0.65 mm in diameter (typically may be between 0.1 mm and 10 mm), and 10 mm in length (typically may be between 0.5 mm and 20 mm). A flow release passage 313 is provided which may become operable when flow through the shut-off is shut off by valve closure member 308 and the valve seat 305. The flow release passage 313 may be any suitable size, for example 0.2 mm in diameter (typically may be between 0.1 mm and 1.5 mm), and 10 mm in length (typically may be between 0.5 mm and 20 mm). Variable resistance flow passages 322 may also be provided that are selectively occluded by the interaction of the valve closure member 308 and the valve seat 307. The variable resistance flow passages 322 may be any suitable size, for example 0.5 mm, and typically may be between 0.1 mm and 2 mm.

The biasing means 309 is selected so as to provide a biasing force (referred to in FIG. 4 as Pi) which is configured to allow the partition member 304 to move to close the valve when the flow rate through the restricted flow passage 310 exceeds a predetermined flow rate.

In use, fluid enters the valve through an inlet in communication with an aspiration tube. The flow via the restrictive flow passage 310 causes a sensing resistance (Rs) which generates a sensing pressure Ps. When Ps exceeds Pi applied by biasing means 309, the partition member 304 is displaced a distance dx. This in turn causes valve closure member 308 to move relative to valve seat 307, creating variable resistance (Rv). The flow release passage 313 provides bypass resistance (Rb) which creates the maximum allowable resistance shunted across Rv, to prevent the valve not passing any fluid at all and Rv becoming infinite due to the valve closure member 308 being located against the valve seat.

The restricted flow passage 310 may be a tube, typically held in a partition member 304 being in the form of a diaphragm or piston. The diaphragm may be elastic or solid to perform the function of the partition member 304. It can also be a rigid disc, flat or conical, suspended with a suspension member 320 much the same as a small speaker cone suspension which can be corrugated or hemispherical as in FIG. 6. The diaphragm may have a biasing means 309 such as a spring acting on it, or have the appropriate elastic properties itself obviating the need for a spring.

The valve closure member 308 and the valve seat 307 may be provided in a form observed in a needle valve, ball valve, poppet valve, hole occlusion valve, or any suitable configuration. A variant is shown in FIG. 6. A small displacement, dx, typically less than 0.3 to 0.5 mm, although it may vary as described above, can control the operation of valve closure member 308 and the valve seat 307 over a large resistance range of 1×109 to 4×1011 or more.

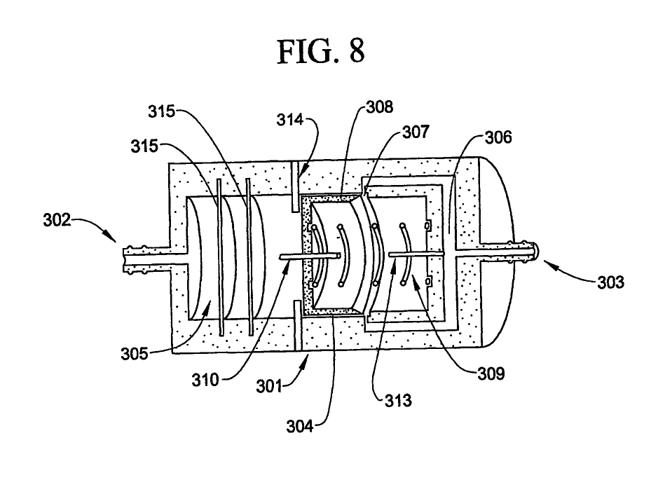

The partition member 304 may also be provided in the form of a piston assembly in which a biasing means 309 is in the form of a return spring. This is shown in FIGS. 7 and 8. Also in this instance, alternative output ports can be taken via hole occlusion valves to alternative output ports 321, as shown in FIG. 7.

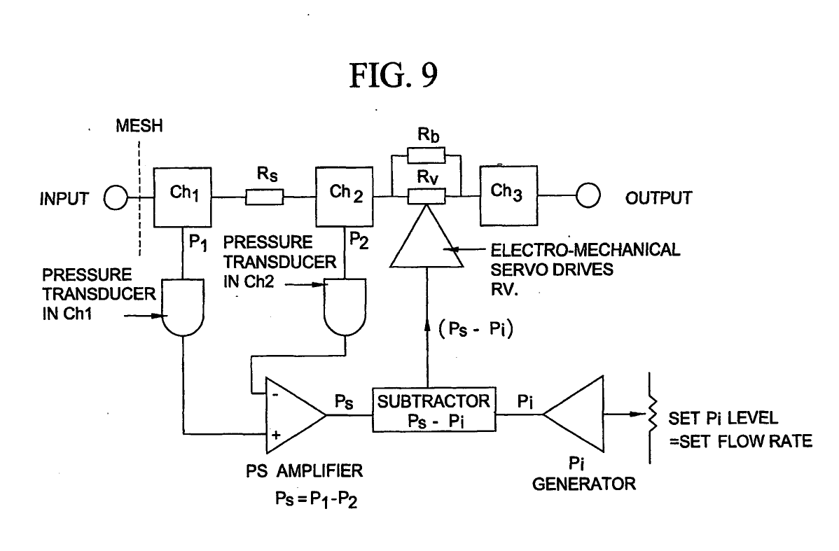

FIG. 9 shows a representation of another embodiment of the valve which is a combination hardware and electronic equivalent designed into the phaco machine's pump system. This can be achieved using electronic pressure sensor means (II) across a flow resistance equivalent to Rs, to generate Ps as an electronic signal, and replacing Rv with an electronic servo driven variable flow resistor. In other words, the system design of FIGS. 6-8 may be implemented in “electromechanical equivalent” form as shown in FIG. 9. In this representation there is a pressure transducer in Chamber 1 and another sensor in chamber 2, Ps would be generated from the difference between the measured pressures from these sensors, Pt would be electronically subtracted from signal Ps and the resultant signal would then control an electromechanical servo device (to regenerate dx) to control a fluid flow resistor Rv, between fluid chambers 2 and 3 in the fluidics system.

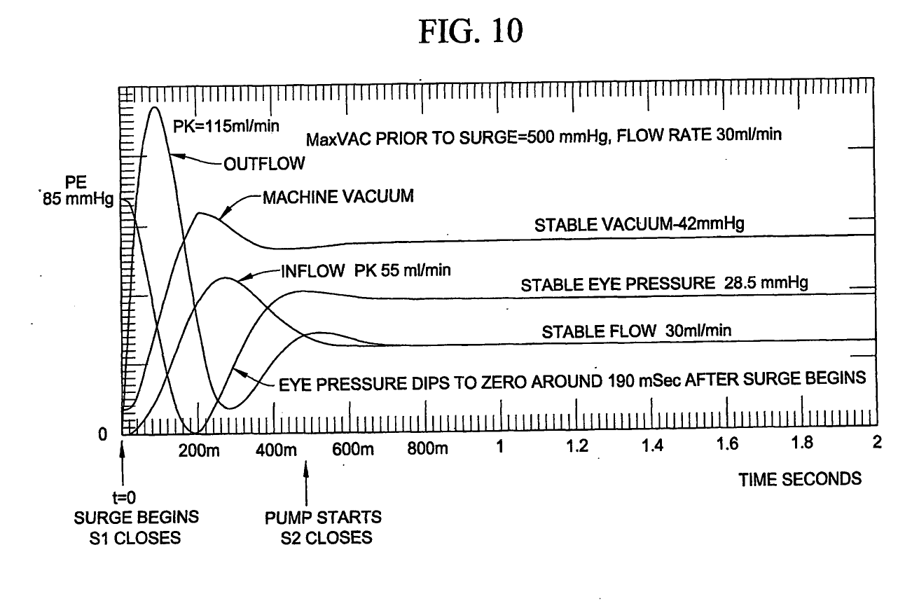

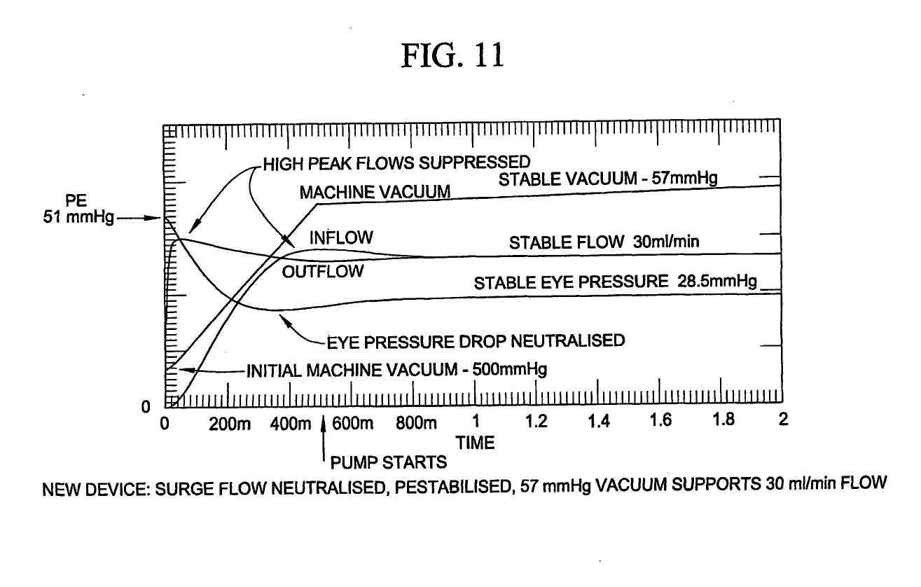

FIGS. 10 to 11 illustrate the pressure conditions that occur in the anterior chamber of the eye in circumstances of a post occlusion surge without the valve (FIG. 10) and with the valve (FIG. 11).

Turning to FIG. 10 there is shown the pressure changes that occur within the anterior chamber of the eye using a Peristaltic Phaco Machine with a typical total system flow resistance (Rt) and a large maximum vacuum (500 mm Hg) to demonstrate the problem of Post Occlusion Surge. Rt is the sum of the total irrigation resistance which includes the flow resistance in the irrigation tubing, the irrigation needle handle and the irrigation needle, and the aspiration flow resistance which includes the resistance in the phaco needle, the probe body supporting the phaco needle and the aspiration tubing that leads to the machines pump.

The amplitude of the negative pressure peak in the eye's anterior chamber is closely proportional to the value of the aspiration vacuum (the vacuum in the aspiration line) prior to the surge occurring.

According to FIG. 10, the occlusion breaks free at time=0 and the pressure dips dramatically to a negative peak at around 190 milliseconds after the surge begins. As can be seen, this drops the anterior chamber pressure from 51 mmHg (its value prior to the surge) down to near zero at 190 milliseconds.

After the surge peaks the pressure returns to a stable value as set by the pumps flow rate F. To support a typical flow rate of 30 ml/min, only a 42 mmHg vacuum is required. In other words, in certain setups, without fixed flow resistors added to the aspiration line, good flow is maintained at low vacuum levels.

FIG. 10 also shows the transient fluid inflow and outflow from the eye and these have significant peaks. The fluid outflow leads in time the pressure drop in the anterior chamber. The fluid inflow is also delayed with respect to the eye pressure drop due to the inertia of the fluid in the irrigation pathways.

The peak outflow value, 115 ml/mm, is very high as shown, and the inflow peak inflow is also high at 55 ml/min but delayed in time. The machine's vacuum collapses rapidly (as shown also on the graph) as fluid enters the aspiration line and the compliant structures expand back to their uncompressed geometry. This is completely unlike the situation where fluid inflow and outflow are identical throughout a steady or equilibrium flow state.

Turning to FIG. 11, the peaks of the fluid inflow and outflow surges to the eye are well suppressed with use of the valve, and as a result the anterior chamber pressure drop is nearly 100% neutralised. After the surge constant flow is re-established and because the control device returns to a low resistance as the vacuum falls, still only a low value of vacuum, 57 mmHg, is required to support a normal flow rate of 30 ml/min which is not high. This is because as the vacuum level has dropped after the surge, and Rv has been adjusted to a much lower value by Ps sensed across Rs acting through dx.

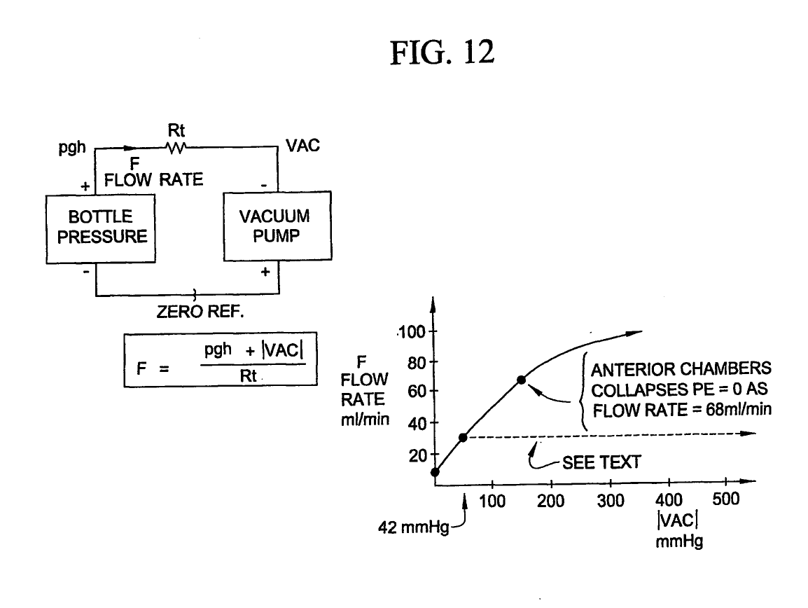

FIG. 12 illustrates the mechanism leading to collapse of the anterior chamber when the flow rate exceeds a specified level. This is of particular relevance to the operation of Venturi based phaco machines in which flow rates cannot be controlled effectively.

FIG. 12 shows that the flow rate is determined by the total of the sum of the bottle pressure and absolute (positive value) of the applied vacuum divided by the total resistance Rt. For example if the vacuum is −42 mmHg and the bottle is 51 mmHg (using pgh of 6798 Nm-2 for a 70 cm bottle height), the driving pressure is 93 mmHg (12396 Newtons/square meter).

A typical Rt (tubing of disposable set with exemplary irrigation line resistance of 6×109 and exemplary aspiration line resistance of 1.88×1010 including and phaco needle irrigating needle etc.) is on the order of 2.47×1010. Therefore 12396/2.47E10=5×10-7 cubic meters/second=a flow rate of 30 ml/min.

The purpose of the solid line graph on FIG. 12 is to show what normally happens when the vacuum increases. If the vacuum is increased to 160 mmHg (and 51 mmHg from the bottle), the driving pressure is now 28126 Newtons/square meter and the flow is now 1.14×10-6 cubic meters/second (68 ml/min) making the anterior chamber pressure zero, even a few more mmHg vacuum collapses the chamber. Some Venturi machines have long aspiration tubing to increase Ra, and also Rt, so vacuums of 200 mmHg may be run before chamber collapse. Again, however, as a consequence, there are lower flow rates when lower vacuums are used at times during the procedure.

As shown above, the valve allows for the two fundamental fluid flow dilemmas of phaco emulsification cataract surgery to be simultaneously solved. The ability to eliminate transient high flow disturbances (e.g., the post occlusion surge) while also providing unimpeded flow at low vacuum levels. In addition the device allows the users of phaco emulsification machines, of any pump type, Peristaltic or Venturi, to run any high level of vacuum they choose, e.g. up to the value which most machines can generate which is around 600 mmHg, without the risk of anterior chamber collapse during the surgery. Higher vacuums are advantageous in efficiently aspirating cataract material from the eye at certain times during the cataract extraction procedure, but lower vacuums are safer at other times, and at those times the flow rate needs to be maintained. The valve device results in better lens fragment holding power and aspiration power of the lens fragments at the tip of the phaco needle and safer and more efficient cataract extraction without the risk of anterior chamber collapse and wound burns.

The valve may be a disposable item, as depicted in FIG. 3 or 6-8, of low cost, which can be added to any existing phaco machine, by placing it in the machine's aspiration tubing near the machines pump or cassette. Alternatively this device can also be built into any manufacturer's existing cassette/disposables system to improve the fluidics performance of them.

The invention has been described with reference to particular embodiments. However, it will be readily apparent to those skilled in the art that it is possible to embody the invention in specific forms other than those of the embodiments described above. The embodiments are merely illustrative and should not be considered restrictive. The scope of the invention is given by the appended claims, rather than the preceding description, and all variations and equivalents which fall within the range of the claims are intended to be embraced therein.

The reader's attention is directed to all papers and documents which are filed concurrently with this specification and which are open to public inspection with this specification, and the contents of all such papers and documents are incorporated herein by reference. All the features disclosed in this specification (including any accompanying claims, abstract, and drawings) may be replaced by alternative features serving the same, equivalent or similar purpose, unless expressly stated otherwise. Thus, unless expressly stated otherwise, each feature disclosed is one example of a generic series of equivalent or similar features.

1. A flow control device comprising: a body having; a chamber formed therein; at least one inlet in communication with the chamber; and at least one outlet in communication with the chamber; wherein the chamber has at least a first portion and at least a second portion that are substantially divided by a member where the member has at least one restricted flow passage, and wherein the member is adapted to adjust a flow rate through the body by adjusting a flow resistance through the body responsive to the flow rate through the restricted flow passage within the device.

2. A flow control device comprising: a body having; a chamber formed therein; at least one inlet in communication with the chamber; and at least one outlet in communication with the chamber; wherein the chamber has at least a first portion and a least a second portion that are divided by a member where the member has at least one restricted flow passage, and wherein the member is adapted to adjust a flow rate through the body by being capable of alternating between a more flow resistance and a less flow resistance configuration in response to flow variations through the device.

3. A flow control device comprising: a body having; a chamber formed therein; means for an inlet of fluid into the chamber; means for outlet out of fluid from the chamber; means for dividing the chamber where the chamber has at least a first portion and at least a second portion; means for restricting the flow of fluid between the at least a first portion and the at least a second portion; and means for adjusting a flow rate through the body responsive to a differential flow rate through the means for restricting the flow of fluid.

4. The flow control device of claim 1 wherein the member is subjected to a biasing force that causes the member to minimize the flow resistance until the flow rate exceeds a predetermined value.

5. The flow control device of claim 4 wherein the biasing force is a spring.

6. The flow control device of claim 5 wherein the biasing force and the member are a diaphragm.

7. The flow control valve of claim 1 wherein the first portion of the chamber has a filter.

8. The flow control device of claim 7 wherein the filter is a mesh filter.

9. The flow control device of claim 8 wherein the filter is made from a synthetic material, a plastic material, or a metallic material or combinations thereof.

10. The flow control device of claim 1 wherein the device body is formed substantially of a polymeric material.

11. The flow control device of claim 1 wherein the body is formed substantially of a silicon material.

12. The flow control device of claim 1 wherein the body is substantially rigid.

13. The flow control device of claim 1 wherein the member is a mechanically movable piston.

14. The flow control device of claim 1 wherein the member is a diaphragm.

15. The flow control device of claim 1 wherein the member is a solenoid operated movable piston.

16. The flow control device of claim 1 wherein the body further comprises: a differential pressure sensor disposed between the inlet and the outlet; and a controller coupled to the differential pressure sensor;
wherein the member is a solenoid operated piston having a position controlled by the controller responsive to a sensed differential pressure.

17. The flow control device of claim 1 wherein the body further comprises: a differential flow sensor disposed between the inlet and the outlet; and a controller coupled to the differential flow sensor; wherein the member is a solenoid operated piston having a position controlled by the controller responsive to a sensed differential flow.

18. The flow control device of claim 1 wherein the outlet has at least one variable resistance orifice and at least one flow bypass orifice in communication with the outlet.

19. The flow control device of claim 18 wherein the member adjusts flow resistance through the body by selectively restricting flow through the at least one variable resistance orifice.

20. The flow control device of claim 1 wherein at least one of the inlet or outlet is sealably attached to an aspiration line.

21. A flow control valve comprising: a valve body having; a chamber formed therein, wherein the chamber is divided by a movable piston into an inlet plenum connected to the inlet and an outlet plenum, said movable piston having an orifice formed there through; an inlet connected between a proximal end of the chamber and an aspiration line by a lure fitting; a filter contained within the inlet; an outlet connected between the distal end of the chamber and the aspiration line by a lure fitting, wherein the outlet plenum has at least one variable resistance orifice and at least one flow bypass orifice connected to the outlet; wherein the movable piston is adapted to adjust flow resistance by selectively changing flow through the at least one variable resistance orifice responsive to a differential pressure between the inlet plenum and the outlet plenum.

22. A method of controlling flow rate through a device comprising the steps of: sensing a flow rate between an inlet and an outlet of a body; if the flow rate increases, adjusting a position of a member such that a flow resistance is increased; and if the flow rate decreases, adjusting the position of the member such that a flow resistance is decreased; whereby an increase in flow rate is countered by an increase in flow resistance and a corresponding mitigation of the increased flow rate, and a decrease in flow rate is countered by a decrease in flow resistance and a corresponding mitigation of the decreased flow rate.

23. A system comprising: a surgical control console; an irrigation device; a surgical instrument for performing an surgical operation on an eye and connected to the irrigation device and the instrument is controlled by the console; an aspiration device connected to the surgical instrument for aspirating fluid and tissue from the eye to a collection receptacle associated with the aspiration device; and a flow control device connected between the aspiration device and the surgical instrument wherein the flow control device includes, a body having a chamber formed therein; an inlet connected to the chamber; and an outlet connected to the chamber; wherein the chamber is divided by a member into at least a first portion and a second portion; wherein the member is adapted to adjust a flow rate through the body by adjusting a flow resistance through the body responsive to a differential pressure between the inlet and the outlet or responsive to a flow rate passing via the device.

24. The system of claim 23 wherein the irrigation device, the surgical, aspiration device, and the flow control device are inter connected with tubing to allow fluid to flow through the system as needed.

25. The system of claim 24 wherein the control flow device and tubing are disposable.

26. The system of claim 23 wherein a set off directions are provided on how to use the control flow device.
